(19) United States
(12) Patent Application Publication
(10) Pub. No.: US 2022/0395443 A1
(43) Pub. Date: Dec. 15, 2022

(54) Oral Care Compositions and Methods

(71) Applicant: Colgate-Palmolive Company, New York, NY (US)

(72) Inventors: Lyndsay SCHAEFFER-KORBYLO, Flemington, NJ (US); Carl MYERS, Wayne, NJ (US); Gokul GOVINDARAJU, Somerville, NJ (US); Reeba PAUL, Edison, NJ (US)

(73) Assignee: Colgate-Palmolive Company, New York, NY (US)

(21) Appl. No.: 17/826,961

(22) Filed: May 27, 2022

Related U.S. Application Data
(60) Provisional application No. 63/193,961, filed on May 27, 2021.

(51) Int. Cl. A61K 8/44; A61K 8/19; A61K 8/24; A61K 8/21; A61K 8/20; A61Q 11/00

(52) U.S. Cl. CPC A61K 8/44; A61K 8/19; A61K 8/24; A61K 8/21; A61K 8/20; A61Q 11/00; A61K 2800/92

(57) ABSTRACT

In one aspect, the application relates to novel aqueous oral care compositions useful for combining and delivering potassium salts and a basic amino acid in a high-water composition, for example, to naturally promote nitrate reduction from the oral microbiome, which can result in systemic increases of nitric oxide in blood plasma and can form part of an overall regimen to maintain, reduce, treat or control systemic blood pressure.

### CROSS-REFERENCE TO RELATED APPLICATIONS

This application claims the benefit of priority to U.S. provisional application 63/193,961, filed May 27, 2021, the entire contents of which are incorporated herein by reference in their entirety.

### FIELD

In one aspect, this application relates to novel aqueous oral care compositions useful for combining and delivering potassium salts and a basic amino acid in a high-water composition, useful in order to naturally promote nitrate reduction from the oral microbiome, which can eventually result in systemic increases of nitric oxide in blood plasma and can form part of an overall regimen to maintain or control systemic blood pressure.

### BACKGROUND

Dental plaque is a sticky biofilm or mass of bacteria that is commonly found between the teeth, along the gum line, and below the gum line margins. Dental plaque can give rise to dental caries and periodontal problems such as gingivitis and periodontitis. Dental caries tooth decay or tooth demineralization caused by acid produced from the bacterial degradation of fermentable sugar. Consequently, the presence of biofilm can be detrimental to the overall health of one's oral cavity. And while oral care is often thought of simply in terms of maintaining oral health and preventing cavities, gingivitis or malodor, the oral cavity also plays a role in the overall health of the body. For example, the mouth serves as an entry point to other organ systems, such as the digestive and circulatory systems, and it has been observed in the field that patients treated with long-term chlorhexidine often experience small, but significant, transient increases in systolic blood pressure.

One way to enhance or improve systemic health, e.g., by improving the health of the oral cavity, is to increase the amount circulating nitric oxide in plasma. In turn, “enterosalivary nitrate cycling” refers to the mechanism whereby dietary nitrate is reduced to nitrite by salivary bacteria. Nitrite which is ingested or absorbed can then be converted to nitric oxide by bacteria in the gut and this nitric oxide can then diffuse into the circulatory system. Plasma nitric oxide can serve as a vasodilator and lead to reductions in blood pressure. Harnessing this potential and promoting the growth and metabolism of salivary nitrate reducing bacteria can lead to meaningful reductions in blood pressure. Consequently, compounds that can decrease biofilm, and potentially increase the amount circulating nitric oxide in an individual's system, could be beneficial in terms of improving both oral and systemic health, e.g., by maintaining or controlling blood pressure.

Accordingly, there is thus a need for novel oral compositions that can benefit systemic health, e.g., by helping to maintain or control blood pressure.

### BRIEF SUMMARY

In one aspect, the oral care compositions described herein contemplate compositions that comprise a soluble nitrate salt (e.g., KNO₃), a basic amino acid (e.g., arginine) in a high-water oral care composition. In one aspect, the compositions function as system for the promotion of enterosalivary nitrate metabolism which can help to reduce, maintain, and/or control blood pressure, e.g., by increasing the levels of nitric oxide in a subject's circulating blood plasma.

In one aspect, and without being bound by theory, the inventors have discovered that KNO₃ and arginine have a surprising effect on the growth of particular nitrate reducing bacterial species, at the expense of non-nitrate reducers, which can then lead to a shift in the overall oral bacterial community to one that overall has increased nitrate reduction capability.

Without being bound by theory, a number of oral bacterial species have been identified as being involved in oral nitrate reduction, and the compositions described herein (e.g., Composition 1.0 et seq) are believed to be able to increase the presence of one or more of oral bacterial species involved in enterosalivary nitrate metabolism. In one aspect, the compositions described herein (e.g., Compositions 1.0 et seq) can increase the presence of one or more of the following bacterial species believed to be involved in enterosalivary nitrate metabolism: Actinomyces naeslundii, Actinomyces odontolyticus, Actinomyces oris, Actinomyces viscosus, Bacillus brevis, Capnocytophaga sputigena, Corynebacterium durum, Corynebacterium matruchotii, Eikenella corrodens, Granulicatella adiacens, Haemophilus parainfluenzae, Haemophilus segnis, Microbacterium oxydans, Neisseria flavescens, Neisseria sicca, Neisseria subflava, Prevotella melaninogenica, Prevotella salivae, Priopionibacterium acnes, Rothia denticariosa, Rothia mucilaginosa, Staphylococcus epidermidis, Staphylococcus hemolyticus, Selenomonas noxia, Veillonella dispar, Veillonella parvula, and Veillonella atypica. Without being bound by theory, it is believed that by increasing the presence of one or more of the oral bacterial species involved in enterosalivary nitrate metabolism, this can eventually contribute to the increase of a subject's plasma nitric oxide levels.

Again, without being bound by theory, the compositions described herein are believed to be able to deliver substrates to oral bacteria, where the substrates are designed to target and promote oral bacteria capable of metabolizing nitrate. In turn, the administration of the compositions described herein (e.g., any of Composition 1.0 et seq) can shift the balance of the oral bacterial community to one where more nitrate reduction occurs, which will lead to increased nitrite being ingested and passed into the gut, and then further reduced to nitric oxide.

It is believed that the community composition of the oral cavity is considerably more stable than other sites of the body and, therefore, repeated, prolonged exposure is required in order to create meaningful bacterial community shifts. The use of oral care formulations described herein allows for delivery of ingredients designed to feed the nitrate reducing bacteria in the oral cavity which allows for repeated application over extended periods of time, and promoting shifts in the oral bacterial community.

The compositions described herein (e.g., any of Composition 1.0 et seq) are believed to provide active ingredients that can naturally promote nitrate reduction from the oral microbiome. For example, potassium salts, such as KNO₃, are believed to provide a short-term source of nitrate to help promote overall nitrate metabolism within the oral bacterial community. Without being bound by theory, a basic amino acid, such as arginine, may serve as a starting substrate in the nitrite reduction pathway that ultimately leads to the production of nitric oxide, the desired endpoint of enterosalivary nitrate cycling. By providing exogenous arginine, for example, and without being bound by theory, the oral care compositions described herein are believed to promote the long-term nitrate reducing capacity of an individual. This, in turn, is believed to lead to increased nitrate cycling and, ultimately, improved blood pressure control via increasing the levels of circulating nitric oxide in the blood plasma.

The disclosure further provides single-component oral care composition packages comprising the compositions disclosed herein.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### DETAILED DESCRIPTION

The following description of the preferred embodiment(s) is merely exemplary in nature and is in no way intended to limit the disclosure, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight of the total composition. Unless otherwise specified, the amounts given are based on the active weight of the material.

As is usual in the art, the compositions described herein are sometimes described in terms of their ingredients, notwithstanding that the ingredients may disassociate, associate or react in the formulation. Ions, for example, are commonly provided to a formulation in the form of a salt, which may dissolve and disassociate in aqueous solution. It is understood that the disclosure encompasses both the mixture of described ingredients and the product thus obtained.

In a first aspect, the present disclosure provides an oral care composition (Composition 1.0) comprising:
(i) a water-soluble nitrate salt (e.g., potassium nitrate);
(ii) a basic amino acid in free or orally acceptable salt form (e.g., arginine);
(iii) water soluble alkali metal polyphosphate (e.g., sodium or potassium pyrophosphate or tripolyphosphate); and
(iii) more than 10% water, by weight of the composition.

For example, the disclosure provides embodiments of Composition 1.0 as follows:
1.1 Composition 1.0, comprising a water-soluble nitrate salt is selected from an alkali or alkaline earth metal nitrate, or zinc nitrate, silver nitrate, or ammonium nitrate.
1.2 Composition 1.1, wherein the water-soluble nitrate salt is an alkali metal nitrate salt or an alkaline earth metal nitrate salt.
1.3 Composition 1.2, wherein the nitrate salt is selected from lithium nitrate, sodium nitrate, potassium nitrate, magnesium nitrate, and calcium nitrate.
1.4 Composition 1.3, wherein the nitrate salt is potassium nitrate.
1.5 Any foregoing composition, wherein the water-soluble alkali metal polyphosphate is selected from a pyrophosphate, tripolyphosphate, tetraphosphate or hexametaphosphate.
1.6 Any foregoing composition, wherein the water-soluble alkali metal polyphosphate is a sodium or potassium polyphosphate.
1.7 Any foregoing composition, wherein the water-soluble alkali metal polyphosphate is selected from sodium pyrophosphate, potassium pyrophosphate, sodium tripolyphosphate and potassium tripolyphosphate.
1.8 Composition 1.7, wherein the sodium pyrophosphate salt is selected from sodium acid pyrophosphate (i.e., disodium pyrophosphate) and tetrasodium pyrophosphate.
1.9 Any foregoing composition, wherein the water-soluble nitrate salt is potassium nitrate and the water-soluble alkali metal polyphosphate salt is tetrasodium pyrophosphate.
1.10 Any foregoing composition, wherein the water-soluble alkali metal polyphosphate alkali phosphate is in the amount from 1.5%-5% by wt. of the composition (e.g., 1.65%-2.25% by wt.) (e.g., tetrasodium pyrophosphate from 1.65%-2.25% by wt.) or in an amount from 0.5%-5% by wt. of the composition (e.g., from 0.5%-1.5% by wt.) (e.g., about 1.2% by wt.);
1.11 Any foregoing composition, wherein the composition comprises a stannous ion source from 0.1 to 2% by wt. of the composition, wherein the stannous ion source is selected from stannous fluoride, stannous chloride or stannous pyrophosphate, or combinations thereof, e.g., 0.1 to 1%, or 0.25 to 0.75%, or about 0.45 by wt. of the composition.
1.12 Any foregoing composition, wherein the composition comprises from 0.1 to 5% by wt. of the nitric acid or water-soluble nitrate salt (e.g., potassium nitrate), by weight of the composition, e.g., 0.1 to 5%, or 1 to 4%, or 2.5 to 3.5% by wt., or about 3% by wt.
1.13 Any foregoing composition, wherein the composition comprises from 1.75 to 5% of the alkali metal polyphosphate salt (e.g., tetrasodium pyrophosphate or sodium tripolyphosphate), by weight of the composition, e.g., 1.75 to 3%, or 1.75 to 2.5%, or 1.75 to 2.25%, or about 2% (e.g., 2% by wt.), or from 0.5%-2% by wt. of the of the alkali metal polyphosphate salt (e.g., tetrasodium pyrophosphate or sodium tripolyphosphate), (e.g., from 0.5%-1.5% by wt.) (e.g., about 1.2% by wt.).
1.14 Any foregoing composition, wherein the composition comprises at least 20% water by weight of the composition, e.g., at least 30%, or at least 40%, or at least 50%, or at least 60% or at least 65%, up to 95% water, by weight of the composition.
1.15 Any foregoing composition, wherein the composition comprises from 10%-90% water by weight of the composition, e.g., 10%-75%, e.g., 10%-60%, e.g., 10% 50%, e.g., 10%-40%.
1.16 Any foregoing composition wherein the composition comprises 70% to 95% water, by weight of the composition, e.g., from 75% to 95%, or from 75% to 90%, or from 75% to 85%, or from 75% to 80%; or wherein the composition comprises from 10% to 50% water, by weight of the composition, e.g., 10% to 40%, or 10% to 30%.
1.17 Any foregoing composition, wherein the composition comprises one or more humectants (e.g., glycerin, sorbitol, propylene glycol, or a mixture thereof) in a net amount of 5% to 70% by weight of the composition, e.g., from 5% to 30% by weight of the composition, or from 10% to 25%, or from 15% to 25%, or about 20%, or from 30 to 70%, or from 30 to 60%, or from 30 to 50%, or from 30% 40% by weight of the composition.
1.18 Any foregoing composition, wherein the composition is a single phase, i.e., it does not form two phases on standing.
1.19 Any foregoing composition, wherein the composition is a clear (e.g., not opaque or turbid) solution (e.g., not a suspension).
1.20 Any foregoing composition, wherein the composition has a pH of between 5 and 9, or a pH between 6 and 8, or a pH between 6.5 and 7.5, or a pH between 6.9 and 7.1, or a pH of about 7.
1.21 Any foregoing composition, wherein the composition comprises less than 10%© of any hydrophobic liquid or mixture of hydrophobic liquids (e.g., alkyl fatty acid esters (e.g., isopropyl myristate), vegetable oils, mineral oils; or combinations thereof), by weight of the composition, for example, less than 5% by weight or less than 3% by weight or less than 1% by weight, of such hydrophobic liquids.
1.22 Any foregoing composition, wherein the composition is free or substantially free of any hydrophobic liquid or mixture of hydrophobic liquids (e.g., less than 0.1% by weight of the composition).
1.23 Any foregoing composition, further comprising a nonionic surfactant, e.g., a hydrophilic nonionic surfactant.
1.24 Any foregoing composition, comprising a nonionic surfactant in an amount of 0.01 to 5.0%, by weight of the composition, e.g., 0.1 to 1.0%, 0.2 to 0.7%, 0.3 to 0.5%, about 0.4%.
1.25 Any foregoing composition, further comprising an anionic surfactant, e.g., selected from sodium laurel ether sulfate (SLES), sodium lauryl sulfate, and ammonium auryl sulfate.
1.26 Any foregoing composition, further comprising an amphoteric surfactant, e.g., selected from: betaines (such as cocamidopropylbetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxylate, sulfonate, sulfate, phosphate or phosphonate), and mixtures thereof.
1.27 Any foregoing composition wherein the composition further comprises one or more of a thickener, a buffer, a sweetener, a flavorant, a pigment, a dye, an anti-caries agent, an anti-bacterial agent, a whitening agent, a desensitizing agent, a preservative, or a mixture thereof.
1.28 Any foregoing composition wherein the composition further comprises an additional fluoride ion source.
1.29 The preceding composition, wherein the additional fluoride ion source is selected from sodium fluoride, potassium fluoride, sodium monofluorophosphate, sodium fluorosilicate, ammonium fluorosilicate, amine fluoride (e.g., N′-octadecyltrimeylendiamine-N,N,N-tris(2-ethanol)-dihydrofluoride), ammonium fluoride, titanium fluoride, hexafluorosulfate, or a mixture thereof.
1.30 Any foregoing composition wherein the composition comprises a whitening agent.
1.31 Any foregoing composition wherein the composition comprises a whitening agent, wherein the whitening agent is hydrogen peroxide.
1.32 Any foregoing composition wherein the composition is the form of a dentifrice (e.g., a toothpaste or a tooth gel), a mouthwash, toothpaste, tooth gel, tooth powder, non-abrasive gel, mousse, foam, mouth spray, lozenge, oral tablet, chewing gum, vitamin, capsule, and dental implement.
1.33 Any foregoing composition, wherein the composition is free of abrasives (e.g., the composition is free of silicas).
1.34 Any foregoing composition, wherein the composition comprises abrasive (e.g., silicas) in an amount of 1-30% by weight of the composition, e.g., 10-30%, or 20-25%.
1.35 Any foregoing composition wherein the basic amino acid is selected from the following: arginine, lysine, serine, citrullene, ornithine, creatine, histidine, diaminobutanoic acid, diaminoproprionic acid, and combinations thereof (e.g., and salts thereof) (e.g., from 1-5% by wt.) (e.g., about 1-3% by wt.) (e.g., 5-15% by wt.) (e.g., about 1.3%) (e.g., about 1.5%).
1.36 The preceding composition, wherein the basic amino acid is arginine (e.g., in free or salt form) (e.g., L-arginine).
1.37 The preceding composition, wherein the amount of arginine is from 1-15% by wt. of the oral care composition. (e.g., from 1-5% by wt.) (e.g., about 1-3% by wt.) (e.g., 5-15% by wt.) (e.g., about 1.3%) (e.g., about 1.5%).
1.38 Any of the foregoing compositions, wherein the composition is effective upon application to the oral cavity, e.g., by rinsing, optionally in conjunction with brushing, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing and/or malodor producing bacteria, (viii) treat, relieve or reduce dry mouth, (ix) clean the teeth and oral cavity, (x) whiten the teeth, (xi) reduce tartar build-up, (xii) reduce or prevent oral malodor, and/or (xiii) promote systemic health, including cardiovascular health, e.g., by reducing potential for systemic infection via the oral tissues.
1.39 Any foregoing composition, wherein the composition is packaged in a container comprising a single storage compartment, which compartment comprises the composition, and a closure (e.g., a screw-top closure) which seals the compartment.
1.40 Any of the foregoing compositions further comprising 0.01 to 0.09% by weight of charcoal (e.g., activated charcoal); wherein the composition is formulated as a dentifrice (e.g., toothpaste or tooth gel).
1.41 Any foregoing compositions, wherein the composition comprises stannous fluoride (e.g., where stannous fluoride is the only source of stannous in the composition).
1.42 Any of the preceding compositions, wherein the composition comprises stannous chloride (e.g., where stannous chloride is the only source of stannous in the composition).
1.43 Any of the preceding compositions, wherein the composition comprises stannous pyrophosphate (e.g., where stannous pyrophosphate is the only source of stannous in the composition).
1.44 Any of the preceding compositions comprising a combination of stannous fluoride and stannous chloride or stannous fluoride and stannous pyrophosphate.
1.45 Any of compositions 1.0-1.44 comprising a combination of one or more of stannous fluoride, stannous chloride, and stannous pyrophosphate.
1.46 Any of the preceding compositions, wherein the composition comprises:
   (i) arginine;
   (ii) potassium nitrate;
   (iii) tetrasodium pyrophosphate from 0.1-2.5% (e.g., about 1.2%) (e.g., about 2%);
   (iv) more than 10% water, by weight of the composition.
1.47 Any of the preceding compositions, wherein the composition comprises:
   (v) arginine;
   (vi) potassium nitrate;
   (vii) tetrasodium pyrophosphate from 1.65%-2.25% by wt. (e.g., about 2%);
   (viii) more than 10% water, by weight of the composition.
1.48 Any of the preceding compositions, wherein the composition comprises:
   (i) 0.1-5% by wt. of potassium nitrate (e.g., about 3%);
   (ii) 1.65-2.25% by wt. of tetrasodium pyrophosphate;
   (iii) 0.1%-5% by wt. of arginine (e.g., about 1.3% by wt.) (e.g., about 1.5% by wt.); and
   (iv) more than 10% water, by weight of the composition (e.g., 10%-90% by wt.)
1.49 Any of the preceding compositions, wherein the composition comprises:
   a.) from 0.1-5% by wt. of potassium nitrate (e.g., about 3%);
   b.) tetrasodium pyrophosphate (e.g., from 1.65-2.25% by wt.);
   c.) stannous fluoride (e.g., from 0.1%-1% by wt.) (e.g., about 0.45%)
   d.) 0.1%-5% by wt. of arginine (e.g., about 1.3% by wt.) (e.g., about 1.5% by wt.); and
   e.) more than 10% water, by weight of the composition (e.g., 10%-90% by wt.)
1.50 Any of the preceding compositions further comprising a zinc ion source selected from zinc oxide, zinc citrate, zinc lactate, zinc phosphate and combinations thereof.
1.51 The preceding composition, wherein the zinc ion source comprises or consists of a combination of zinc oxide and zinc citrate.
1.52 The preceding composition, wherein the ratio of the amount of zinc oxide (e.g., wt. %) to zinc citrate (e.g., wt. %) is from 1.5:1 to 4.5:1 (e.g., 2:1, 2.5:1, 3:1, 3.5:1, or 4:1).
1.53 Either of the two preceding compositions, wherein the zinc citrate is in an amount of from 0.25 to 1.0 wt. % (e.g., 0.5 wt. %) and zinc oxide may be present in an amount of from 0.75 to 1.25 wt. % (e.g., 1.0 wt. %) based on the weight of the oral care composition.
1.54 Any of the preceding compositions, wherein the zinc ion source comprises zinc citrate in an amount of about 0.5 wt. %.
1.55 Any of the preceding compositions, wherein the zinc ion source comprises zinc oxide in an amount of about 1.0 wt. %.
1.56 Any of the preceding compositions, wherein the zinc ion source comprises zinc citrate in an amount of about 0.5 wt. % and zinc oxide in an amount of about 1.0 wt. %.
1.57 Any of the preceding compositions, wherein the oral care composition can be ingested (e.g., intentionally ingested subsequent to consumption).
1.58 Any of the preceding compositions, wherein the composition comprises a taurate surfactant (e.g., sodium methyl cocoyl taurate).
1.59 Any of the preceding compositions, wherein the composition is free of any of sodium lauryl sulfate.
1.60 Any of the preceding compositions, wherein the composition comprises:
   (i) Arginine (e.g., from 0.5%-6% by wt.) (e.g., about 1.3%) (e.g., about 1.5%) (e.g., about 5% by wt.);
   (ii) potassium nitrate (e.g., from 0.5%-7.5% by wt.);
   (iii) tetrasodium pyrophosphate from 0.1-3% by wt. (e.g., about 1.2%) (e.g., about 2%);
   (iv) a stannous ion source comprises a stannous ion source selected from: stannous fluoride, stannous chloride, stannous pyrophosphate, and combinations thereof; and
   (v) more than 10% water, by weight of the composition.
1.61 The oral care composition of the preceding composition, wherein the stannous ion source comprises stannous fluoride.

In a second aspect, the present disclosure further provides a method (Method 1) of treating or reducing blood pressure (e.g., systemic blood pressure), wherein the method comprises administration of a composition according to any of Composition 1.0 et seq (e.g., any of Composition 1.0-1.61), to the oral cavity of a subject in need thereof, e.g., by brushing, for example, one or more times per day.

For example, the disclosure provides embodiments of Method 1.0 as follows:
1.1 Method 1.0, wherein the subject in need thereof has elevated blood pressure and/or is at risk for elevated blood pressure.
1.2 Method 1.0 or 1.1 wherein the method is administered to a subject in need thereof in order to increase the presence of one or more oral bacteria in the oral cavity.
1.3 Any of Method 1.0-1.2, wherein the oral bacteria is selected from: Actinomyces naeslundii, Actinomyces odontolyticus, Actinomyces oris, Actinomyces viscosus, Bacillus brevis, Capnocytophaga sputigena, Corynebacterium durum, Corynebacterium matruchotii, Eikenella corrodens, Granulicatella adiacens, Haemophilus parainfluenzae, Haemophilus segnis, Microbacterium oxydans, Neisseria flavescens, Neisseria sicca, Neisseria subflava, Prevotella melaninogenica, Prevotella salivae, Priopionibacterium acnes, Rothia denticariosa, Rothia mucilaginosa, Staphylococcus epidermidis, Staphylococcus hemolyticus, Selenomonas noxia, Veillonella dispar, Veillonella parvula, Veillonella atypica, and combinations thereof.

1.4 The preceding method, wherein the administration of any of Composition 1.0 et seq increase the presence of a bacteria selected from: Prevotella melaninogenica, Veillonella dispar, Haemophilus parainfluenzae, Neisseria subflava, Veillonella parvula, Rothia mucilaginosa Rothia dentocariosa, Actinomyces viscosus and combinations thereof
1.5 The preceding method wherein Veillonella parvula is increased.
1.6 The method of 1.4, wherein Neisseria subflava is increased.
1.7 The method of 1.4, wherein Haemophilus parainfluenzae is increased.
1.8 The method of 1.4, wherein Rothia dentocariosa is increased.
1.9 Any of the preceding methods, wherein the subject in need thereof has low or reduced amounts of oral bacteria selected from: Actinomyces naeslundii, Actinomyces odontolyticus, Actinomyces oris, Actinomyces viscosus, Bacillus brevis, Capnocytophaga sputigena, Corynebacterium durum, Corynebacterium matruchotii, Eikenella corrodens, Granulicatella adiacens, Haemophilus parainfluenzae, Haemophilus segnis, Microbacterium oxydans, Neisseria flavescens, Neisseria sicca, Neisseria subflava, Prevotella melaninogenica, Prevotella salivae, Priopionibacterium acnes, Rothia denticariosa, Rothia mucilaginosa, Staphylococcus epidermidis, Staphylococcus hemolyticus, Selenomonas noxia, Veillonella dispar, Veillonella parvula, Veillonella atypica, and combinations thereof; and wherein the amounts of oral bacteria are low or reduced relative to a reference standard.
1.10 Any of the preceding methods, wherein the purpose of the administration of any of Composition 1.0, et seq is to deliver substrates to bacteria in the oral cavity.
1.11 The preceding method, wherein the substrates are administered to target and promote oral bacteria capable of metabolizing nitrate.
1.12 Any of the preceding methods, wherein the subject in need thereof has low or reduced amounts of circulating nitric oxide, wherein the amount of circulating nitric oxide is low relative to a reference standard.
1.13 Any of the preceding methods, wherein administration of any of Composition 1.0 et seq, to the subject's oral cavity increases the amount of nitric oxide in the patient's blood plasma.
1.14 Any of the preceding methods comprising brushing with any of Composition 1.0 et seq once a day for five consecutive days.
1.15 Any of the preceding methods wherein the administration with any of Composition 1.0 et seq treats or reduces systemic blood pressure.
1.16 Any of the preceding methods wherein the subject's blood pressure is maintained or controlled, e.g., the result of the treatment is that there is no further increase in the subject's systemic blood pressure.
1.17 Any of the preceding methods, wherein the composition that is administered comprises:
   arginine in free or orally acceptable salt form (e.g., L-arginine) (e.g., from 1.25%-1.6% by wt.);
   potassium nitrate;
   tetrasodium pyrophosphate; and
   more than 10% water, by weight of the composition.
1.18 Any of the preceding methods, wherein the composition that is administered comprises:
   arginine in free or orally acceptable salt form (e.g., L-arginine) (e.g., from 1.25%-1.6% by wt.);
   potassium nitrate;
   tetrasodium pyrophosphate from 1.65%-2.25% by wt. (e.g., about 2%); and
   more than 10% water, by weight of the composition.
1.19 Any of the preceding methods, wherein the composition that is administered (e.g., any of Composition 1.0-1.61) is in the form selected from: a mouthwash, a toothpaste, a tooth gel, a tooth powder, a non-abrasive gel, a mousse, a foam, a mouth spray, a lozenge, an oral tablet, vitamin, capsule, and a dental implement.
1.20 Any of the preceding methods, wherein the oral care composition can be ingested as part of the method (e.g., intentionally ingested subsequent to consumption).
1.21 Any of the preceding methods, wherein the method is directed to treating or reducing systemic blood pressure.
1.22 Any of the preceding methods, wherein the composition that is administered comprises:
   (i) Arginine (e.g., from 0.5%-6% by wt.) (e.g., about 1.3%) (e.g., about 1.5%) (e.g., about 5% by wt.);
   (ii) potassium nitrate (e.g., from 0.5%-7.5% by wt.);
   (iii) tetrasodium pyrophosphate from 0.1-3% by wt. (e.g., about 1.2%) (e.g., about 2%);
   (iv) a stannous ion source comprises a stannous ion source selected from: stannous fluoride, stannous chloride, stannous pyrophosphate, and combinations thereof; and
   (v) more than 10% water, by weight of the composition.
1.23 The method of 1.22, wherein the stannous ion source comprises stannous fluoride.
1.24 The method of any of the preceding methods, wherein the oral care composition can be ingested.

In another aspect, the present disclosure provides an oral care package comprising a composition according to Composition 1.0 et seq or Composition 2.0 et seq, wherein the package comprises a container comprising a single storage compartment, which compartment contains the composition, and a closure (e.g., a screw-top closure) which seals the compartment.

In still another aspect, the present disclosure provides a method of treatment or prevention of gingivitis, plaque, dental caries, and/or dental hypersensitivity, the method comprising the application to the oral cavity of a person in need thereof, of a composition according to the disclosure (e.g., Composition 1.0 et seq or Composition 2.0 et seq), e.g., by brushing, for example, one or more times per day.

Alternatively, the present disclosure provides Composition 1.0 et seq or Composition 2.0 et seq, for use in the treatment or prevention of gingivitis, plaque, dental caries, and/or dental hypersensitivity.

In still another aspect, the present disclosure provides Composition 1.0 et seq or Composition 2.0 et seq, for use in the treating or reducing systemic blood pressure (e.g., for use in any of Method 1.0 et seq).

Method 1.0 et seq further comprise applying any of the compositions as described herein to the teeth, e.g., by brushing, gargling or rinsing, or otherwise administering the compositions to the oral cavity of a subject in need thereof. The compositions can be administered regularly, such as, for example, one or more times per day (e.g., twice per day). In various embodiments, administering the compositions of the present disclosure to teeth may provide one or more of the following specific benefits: (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing and/or malodor producing bacteria, (viii) treat, relieve or reduce dry mouth, (ix) clean the teeth and oral cavity, (x) whiten the teeth, (xi) reduce tartar build-up, (xii) reduce or prevent oral malodor, and/or (xiii) promote systemic health, including cardiovascular health, e.g., by reducing potential for systemic infection via the oral tissues.

In yet another aspect, the disclosure provides for a composition (Composition 2.0) comprising:
(i) a water-soluble nitrate salt (e.g., potassium nitrate);
(ii) a basic amino acid in free or orally acceptable salt form (e.g., arginine) and/or neutral amino acid in free orally acceptable salt form (e.g., glycine);
(iii) water soluble alkali metal polyphosphate (e.g., sodium or potassium pyrophosphate or tripolyphosphate); and
(iv) more than 10% water, by weight of the composition.

In yet another aspect, Composition 2.0 can further comprise any of Composition 1.1-1.61 as previously described herein.

In yet another aspect, Composition 2.0 can be administered as part of any of Method 1.0-1.23.

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. In some embodiments, an oral care composition can be intentionally swallowed or ingested at some point following consumption. The oral care compositions as disclosed herein may be used in nonhuman mammals such as companion animals (e.g., dogs and cats), as well as by humans. In some embodiments, the oral care compositions as disclosed herein are used by humans. Oral care compositions include, for example, dentifrice and mouthwash. In some embodiments, the disclosure provides mouthwash formulations.

As used herein, “orally acceptable salt” refers to salts or derivatives used in the present disclosure that are safe for use in the amounts and concentrations provided. Suitable salts include salts known in the art to be pharmaceutically acceptable salts are generally considered to be physiologically acceptable in the amounts and concentrations provided herein. Physiologically acceptable salts include those derived from pharmaceutically acceptable inorganic or organic acids or bases. These include, for example, acid addition salts such as hydrochloride or bromide salt, and base addition such as those derived from alkali metals such as potassium and sodium, or alkaline earth metals such as calcium and magnesium. One example of a salt is bicarbonate (e.g., arginine bicarbonate).

As used herein, “single component” means an oral care composition comprising at most a single compositional component at any time. Thus, this is in distinction to a “dual-component” compositions, which is manufactured as two separate compositions, maintained separately until final point of use. For example, a dual component toothpaste is typically packaged in a tube containing two parallel compartments exiting via a common nozzle such that when the user extrudes the toothpaste from the package the two components mix immediately prior to application to the oral cavity. Likewise, a dual component mouthwash is typically packaged in a bottle comprising two compartments such that a measured amount of the liquid from each compartment is dispensed and mixed when the user. Dual component compositions are often used to maintain in separate components and compartments ingredients which are mutually incompatible, such that if kept in the same component they would adversely react or interfere with each other.

In contrast, a dual-phase composition, such as a mouthwash, is a single-component composition comprising two immiscible liquids which settle into two phases on standing. Such a composition has no need for separated compartments for storage because the natural tendency of the two phases to separate helps ensure that the ingredients in one phase are not maintained in intimate contact with the ingredients of the other phase. Nevertheless, when vigorously mixed, the two phases become intimately combined (such as, to form an emulsion), which may or may not separate back into the two phases on standing.

### Fluoride Ion Source

The oral care compositions may further include one or more fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., each of which are incorporated herein by reference. Representative fluoride ion sources used with the present disclosure (e.g., Composition 1.0 et seq, or Method 1.0 et seq, or Composition 2.0 et seq) include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof. Where the formulation comprises calcium salts, the fluoride salts are preferably salts wherein the fluoride is covalently bound to another atom, e.g., as in sodium monofluorophosphate, rather than merely ionically bound, e.g., as in sodium fluoride.

### Surfactants

The disclosure may in some embodiments contain anionic surfactants, e.g., the (e.g., any of Composition 1.0 et seq, or any of Method 1.0 et seq, or any of Composition 2.0 et seq), for example, water-soluble salts of higher fatty acid monoglyceride monosulfates, such as the sodium salt of the monosulfated monoglyceride of hydrogenated coconut oil fatty acids, such as sodium N-methyl N-cocoyl taurate, sodium cocomo-glyceride sulfate; higher alkyl sulfates, such as sodium lauryl sulfate; higher alkyl-ether sulfates, e.g., of formula CH₃(CH₂)ₘCH₂(OCH₂CH₂)ₙOSO₃X, wherein m is 6-16, e.g., 10, n is 1-6, e.g., 2, 3 or 4, and X is Na or, for example sodium laureth-2 sulfate (CH₃(CH₂)₁₀CH₂(OCH₂CH₂)₂OSO₃Na); higher alkyl aryl sulfonates such as sodium dodecyl benzene sulfonate (sodium lauryl benzene sulfonate); higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. By “higher alkyl” is meant, e.g., C₆₋₃₀ alkyl. In particular embodiments, the anionic surfactant (where present) is selected from sodium lauryl sulfate and sodium ether lauryl sulfate. When present, the anionic surfactant is present in an amount which is effective, e.g., >0.001% by weight of the formulation, but not at a concentration which would be irritating to the oral tissue, e.g., 1%, and optimal concentrations depend on the particular formulation and the particular surfactant. In one embodiment, the anionic surfactant is present at from 0.03% to 5% by weight, e.g., about 1.75% by wt.

In another embodiment, cationic surfactants useful in the present disclosure can be broadly defined as derivatives of aliphatic quaternary ammonium compounds having one long alkyl chain containing 8 to 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof. Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants of the disclosure (e.g., any of Composition 1.0 et seq or any of Method 1.0 et seq, or any of Composition 2.0 et seq) can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials. In a particular embodiment, the composition of the disclosure comprises a nonionic surfactant selected from polaxamers (e.g., polaxamer 407), polysorbates (e.g., polysorbate 20), polyoxyl hydrogenated castor oils (e.g., polyoxyl 40 hydrogenated castor oil), and mixtures thereof.

Illustrative amphoteric surfactants for use in the compositions of the disclosure, e.g., any of Composition 1.0 et seq, or any of Method 1.0 et seq, or any of Composition 2.0 et seq), that can be used in the compositions of the disclosure include betaines (such as cocamidopropylbetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxylate, sulfonate, sulfate, phosphate or phosphonate), and mixtures of such materials.

The surfactant or mixtures of compatible surfactants can be present in the compositions of the present disclosure in 0.1% to 5%, in another embodiment 0.3% to 3% and in another embodiment 0.5% to 2% by weight of the total composition.

### Flavoring Agents

The oral care compositions of the disclosure may also include a flavoring agent. Flavoring agents which are used in the practice of the present disclosure include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint.

The flavoring agent is incorporated in the oral composition at a concentration of 0.01 to 1% by weight.

### pH Adjusting Agents

In some embodiments, the compositions of the present disclosure contain a buffering agent. Examples of buffering agents include anhydrous carbonates such as sodium carbonate, sesquicarbonates, bicarbonates such as sodium bicarbonate, silicates, bisulfates, phosphates (e.g., monopotassium phosphate, monosodium phosphate, disodium phosphate, dipotassium phosphate, tribasic sodium phosphate, sodium tripolyphosphate, pentapotassium tripolyphosphate, phosphoric acid), citrates (e.g. citric acid, trisodium citrate dehydrate), pyrophosphates (sodium and potassium salts, e.g., tetrapotassium pyrophosphate) and combinations thereof. The amount of buffering agent is sufficient to provide a pH of about 5 to about 9, preferable about 6 to about 8, and more preferable about 7, when the composition is dissolved in water, a mouthrinse base, or a toothpaste base. Typical amounts of buffering agent are about 5% to about 35%, in one embodiment about 10% to about 30%, in another embodiment about 15% to about 25%, by weight of the total composition.

### Chelating and Anti-Calculus Agents

The oral care compositions of the disclosure also may include one or more chelating agents able to complex calcium found in the cell walls of the bacteria. Binding of this calcium weakens the bacterial cell wall and augments bacterial lysis.

Another group of agents suitable for use as chelating or anti-calculus agents in the present disclosure are the soluble pyrophosphates. The pyrophosphate salts used in the present compositions can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The salts are useful in both their hydrated and unhydrated forms. An effective amount of pyrophosphate salt useful in the present composition is generally enough to provide at least 0.1 wt. % pyrophosphate ions, e.g., 0.1 to 3 wt. %, e.g., 0.1 to 2 wt. %, e.g., 0.1 to 1 wt. %, e.g., 0.2 to 0.5 wt. %. The pyrophosphates also contribute to preservation of the compositions by lowering water activity.

Suitable anticalculus agents for the compositions of the disclosure (e.g., any of Composition 1.0 et seq, or any of Method 1.0 et seq, or any of Composition 2.0 et seq) include without limitation phosphates and polyphosphates (for example pyrophosphates), polyaminopropanesulfonic acid (AMPS), hexametaphosphate salts, zinc citrate trihydrate, polypeptides, polyolefin sulfonates, polyolefin phosphates, diphosphonates. In particular embodiments, the disclosure includes alkali phosphate salts, i.e., salts of alkali metal hydroxides or alkaline earth hydroxides, for example, sodium, potassium or calcium salts. “Phosphate” as used herein encompasses orally acceptable mono- and polyphosphates, for example, P₁₋₆ phosphates, for example monomeric phosphates such as monobasic, dibasic or tribasic phosphate; dimeric phosphates such as pyrophosphates; and multimeric phosphates, e.g., sodium hexametaphosphate. In particular examples, the selected phosphate is selected from alkali dibasic phosphate and alkali pyrophosphate salts, e.g., selected from sodium phosphate dibasic, potassium phosphate dibasic, dicalcium phosphate dihydrate, calcium pyrophosphate, tetrapotassium pyrophosphate, sodium tripolyphosphate, and mixtures of any of two or more of these. In a particular embodiment, for example the compositions comprise a mixture of tetrasodium pyrophosphate (Na₄P₂O₇), calcium pyrophosphate (Ca₂P₂O₇), and sodium phosphate dibasic (Na₂HPO₄), e.g., in amounts of ca. 3-4% of the sodium phosphate dibasic and ca. 0.2-1% of each of the pyrophosphates. Such phosphates are provided in an amount effective to reduce erosion of the enamel, to aid in cleaning the teeth, and/or to reduce tartar buildup on the teeth, for example in an amount of 2-20%, e.g., ca. 5-15%, by weight of the composition.

### Polymers

The oral care compositions of the disclosure (e.g., any of Composition 1.0 et seq, or any of Method 1.0 et seq, or any of Composition 2.0 et seq) also optionally include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water-soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139(M.W. 500,000), AN 1 19 (M.W. 250,000) and S-97 Pharmaceutical Grade (M.W. 70,000), of GAF Chemicals Corporation.

Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

The N-vinyl-2-pyrrolidione is also commonly known as polyvinylpyrrolidone or “PVP”. PVP refers to a polymer containing vinylpyrrolidone (also referred to as N-vinylpyrrnlidone and N-vinyl-2-pyrrolidinone) as a monomeric unit. The monomeric unit consists of a polar imide group, four non-polar methylene groups and a non-polar methane group. The polymers include soluble and insoluble homopolymeric PVPs. Copolymers containing PVP include vinylpyrrolidone/vinyl acetate (also known as Copolyvidone, Copolyvidonum or VP-VAc) and vinyl pyrrolidone/dimethylamino-ethylmethacrylate. Soluble PVP polymers among those useful herein are known in the art, including Povidone, Polyvidone, Polyvidonum, poly(N-vinyl-2-pyrrolidinone), poly (N-vinylbutyrolactam), poly(-vinyl-2-pyrrolidone) and poly [1-(2-oxo-1 pyrrolidinyl)ethylene]. These PVP polymers are not substantially cross-linked. In some embodiments the polymer comprises an insoluble cross-linked homopolymer. Such polymers include crosslinked PVP (often referred to as cPVP, polyvinylpolypyrrolidone, or cross-povidone).

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000, described in U.S. Pat. No. 4,842,847, Jun. 27, 1989 to Zahid, incorporated herein by reference.

In preparing oral care compositions, it is sometimes necessary to add some thickening material to provide a desirable consistency or to stabilize or enhance the performance of the formulation. In certain embodiments, the thickening agents are carboxyvinyl polymers, carrageenan, xanthan, hydroxyethyl cellulose and water-soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In certain embodiments, thickening agents in an amount of about 0.5% to about 5.0% by weight of the total composition are used.

In some embodiments, microcrystalline cellulose (MCC) can be used (e.g., carboxymethyl cellulose with sodium carboxymethyl cellulose). An example of a source of MCC is Avicel® (FMC Corporation), which contains MCC in combination with sodium carboxymethyl cellulose (NaCMC). Both Avicel®. RC-591 (MCC containing 8.3 to 13.8 weight % NaCMC) and Avicel®. CL-611 (MCC containing 11.3 to 18.8 weight % NaCMC) may be used in certain aspects. In certain embodiments, the ratio of microcrystalline cellulose to cellulose ether thickening agent is from 1:1 to 1:3 by weight; or from 1:1.5 to 1:2.75 by weight. In any of the above embodiments comprising sodium carboxymethylcellulose, microcrystalline cellulose may be used in combination with NaCMC. In certain such embodiments, the MCC/sodium carboxymethylcellulose may be present in an amount of from 0.5 to 1.5 weight % based on the total weight of the composition.

### Abrasives

The compounds of the disclosure (e.g., any of Composition 1.0 et seq, or any of Method 1.0 et seq, or any of Composition 2.0 et seq) may optionally comprise an abrasive. Natural calcium carbonate is found in rocks such as chalk, limestone, marble and travertine. It is also the principle component of egg shells and the shells of mollusks. The natural calcium carbonate abrasive of the disclosure is typically a finely ground limestone which may optionally be refined or partially refined to remove impurities. For use in the present disclosure, the material has an average particle size of less than 10 microns, e.g., 3-7 microns, e.g., about 5.5 microns. For example a small particle silica may have an average particle size (D50) of 2.5-4.5 microns. Because natural calcium carbonate may contain a high proportion of relatively large particles of not carefully controlled, which may unacceptably increase the abrasivity, preferably no more than 0.01%, preferably no more than 0.004% by weight of particles would not pass through a 325 mesh. The material has strong crystal structure, and is thus much harder and more abrasive than precipitated calcium carbonate. The tap density for the natural calcium carbonate is for example between 1 and 1.5 g/cc, e.g., about 1.2 for example about 1.19 g/cc. There are different polymorphs of natural calcium carbonate, e.g., calcite, aragonite and vaterite, calcite being preferred for purposes of this disclosure. An example of a commercially available product suitable for use in the present disclosure includes Vicron® 25-11 FG from GMZ.

Precipitated calcium carbonate is generally made by calcining limestone, to make calcium oxide (lime), which can then be converted back to calcium carbonate by reaction with carbon dioxide in water. Precipitated calcium carbonate has a different crystal structure from natural calcium carbonate. It is generally more friable and more porous, thus having lower abrasivity and higher water absorption. For use in the present disclosure, the particles are small, e.g., having an average particle size of 1-5 microns, and e.g., no more than 0.1%, preferably no more than 0.05% by weight of particles which would not pass through a 325 mesh. The particles may for example have a D50 of 3-6 microns, for example 3.8=4.9, e.g., about 4.3; a D50 of 1-4 microns, e.g., 2.2-2.6 microns, e.g., about 2.4 microns, and a D10 of 1-2 microns, e.g., 1.2-1.4, e.g., about 1.3 microns. The particles have relatively high-water absorption, e.g., at least 25 g/100 g, e.g., 30-70 g/100 g. Examples of commercially available products suitable for use in the present disclosure include, for example, Carbolag® 15 Plus from Lagos Industria Quimica.

In certain embodiments the disclosure (e.g., any of Composition 1.0 et seq., or Method 1.0 et seq., or any of Composition 2.0 et seq) may comprise additional calcium-containing abrasives, for example calcium phosphate abrasive, e.g., tricalcium phosphate (Ca₃(PO₄)₂), hydroxyapatite (Ca₁₀(PO₄)₆(OH)₂), or dicalcium phosphate dihydrate (CaHPO₄.2H₂O, also sometimes referred to herein as DiCal) or calcium pyrophosphate, and/or silica abrasives, sodium metaphosphate, potassium metaphosphate, aluminum silicate, calcined alumina, bentonite or other siliceous materials, or combinations thereof. Any silica suitable for oral care compositions may be used, such as precipitated silicas or silica gels. For example, synthetic amorphous silica. Silica may also be available as a thickening agent, e.g., particle silica. For example, the silica can also be small particle silica (e.g., Sorbosil AC43 from PQ Corporation, Warrington, United Kingdom). However, the additional abrasives are preferably not present in a type or amount so as to increase the RDA of the dentifrice to levels which could damage sensitive teeth, e.g., greater than 130.

### Amino Acids

The compositions of the disclosure include an amino acid. For example, any of Compositions 1.0 et seq or Method 1.0 et seq can include a basic amino acid. The basic amino acids which can be used in the compositions and methods of the disclosure include not only naturally occurring basic amino acids, such as arginine, lysine, and histidine, but also any basic amino acids having a carboxyl group and an amino group in the molecule, which are water-soluble and provide an aqueous solution with a pH of 7 or greater.

For example, basic amino acids include, but are not limited to, arginine, lysine, serine, citrullene, ornithine, creatine, histidine, diaminobutanoic acid, diaminoproprionic acid, salts thereof or combinations thereof. In a particular embodiment, the basic amino acids are selected from arginine, citrullene, and ornithine.

In certain embodiments, the basic amino acid is arginine, for example, L-arginine, or a salt thereof.

In another aspect, in addition to the basic amino acid included in the formulation, the compositions of the disclosure (e.g., any of Compositions 1.0 et seq, or Method 1.0 et seq, or Composition 2.0 et seq) can include a neutral amino acid, which can include, but are not limited to, one or more neutral amino acids selected from the group consisting of alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and combinations thereof.

In one aspect, the compositions of the disclosure (e.g., any of Composition 1.0 et seq or Method 1.0 et seq, or Composition 2.0 et seq) are intended for topical use in the mouth and so salts for use in the present disclosure should be safe for such use, in the amounts and concentrations provided. Suitable salts include salts known in the art to be pharmaceutically acceptable salts are generally considered to be physiologically acceptable in the amounts and concentrations provided. Physiologically acceptable salts include those derived from pharmaceutically acceptable inorganic or organic acids or bases, for example acid addition salts formed by acids which form a physiological acceptable anion, e.g., hydrochloride or bromide salt, and base addition salts formed by bases which form a physiologically acceptable cation, for example those derived from alkali metals such as potassium and sodium or alkaline earth metals such as calcium and magnesium. Physiologically acceptable salts may be obtained using standard procedures known in the art, for example, by reacting a sufficiently basic compound such as an amine with a suitable acid affording a physiologically acceptable anion.

### Water

Water is present in the oral compositions of the disclosure. Water, employed in the preparation of commercial oral compositions should be deionized and free of organic impurities. Water commonly makes up the balance of the compositions and includes 5% to 45%, e.g., 10% to 20%, e.g., 25-35%, by weight of the oral compositions. This amount of water includes the free water which is added plus that amount which is introduced with other materials such as with sorbitol or silica or any components of the disclosure. The Karl Fischer method is a one measure of calculating free water.

### Humectants

Within certain embodiments of the oral compositions, it is also desirable to incorporate a humectant to reduce evaporation and also contribute towards preservation by lowering water activity. Certain humectants can also impart desirable sweetness or flavor to the compositions. The humectant, on a pure humectant basis, generally includes 15% to 70% in one embodiment or 30% to 65% in another embodiment by weight of the composition.

Suitable humectants include edible polyhydric alcohols such as glycerin, sorbitol, xylitol, propylene glycol as well as other polyols and mixtures of these humectants. Mixtures of glycerin and sorbitol may be used in certain embodiments as the humectant component of the compositions herein.

Flavorings for use in the present disclosure may include extracts or oils from flavorful plants such as peppermint, spearmint, cinnamon, wintergreen, and combinations thereof, cooling agents such as menthol, methyl salicylate, as well as sweeteners, which may include polyols (which also function as humectants), saccharin, acesulfame, aspartame, neotame, stevia and sucralose.

### EXAMPLES

Unless otherwise noted, the pH of all solutions described in the Examples is about 7. Unless otherwise noted, all figures for stannous ion concentration refer to soluble stannous, not total stannous (total stannous being soluble and insoluble stannous combined).

### Example 1—Dentifrice Formulations

Exemplary representative dentifrice compositions according to the present disclosure are expected to be formulated as follows (quantities shown in % by weight of the composition):

| | Formula | Control | Control |
|---|---|---|---|
| | A | 1 | 2 |
| Ingredients | Paste | Paste | Paste |
| Water | Q.S. | Q.S. | Q.S. |
| | (eg, | (eg, | (eg, |
| | ~21) | ~21) | ~21) |
| L-Arginine | 1.5 | — | 1.5 |
| Polyethylene glycol (e.g., | 2 | 2 | 2 |
| PEG 600) | | | |
| Xanthan Gum | 0.3 | 0.3 | 0.3 |
| Carboxymethyl cellulose | 0.8 | 0.8 | 0.8 |
| (e.g., Na CMC) | | | |
| Stannous Fluoride | 0.454 | 0.454 | 0.454 |
| Potassium Nitrate | 3 | 3 | — |
| Tetrasodium | 2 | 2 | 2 |
| Pyrophosphate | | | |
| Glycerin | 6 | 6 | 6 |
| Sorbitol (70% sol.) | 37.3 | 38.8 | 40.3 |
| Silicas | 21.5 | 21.5 | 21.5 |
| Anionic surfactant (e.g., | 1.5 | 1.5 | 1.5 |
| SLS) | | | |
| Zwitterionic surfactant | 1.25 | 1.25 | 1.25 |
| (e.g., betaine) | | | |
| Flavors, Colors, and other | 1.3 | 1.3 | 1.3 |
| Minors | | | |
| Total | 100 | 100 | 100 |

### Example 2—Growth of Nitrate Reducing Species in Optical Density Assay

The impact of 1.5% KNO₃ and 0.75% L-arginine alone or in combination on the growth of one known nitrate reducing species, Haemophilus parainfluenzae, is studied. Pure cultures of each species are grown in the presence of the compounds. Note, the wt. % of the potassium nitrate and L-arginine is relative to the total weight of the solution added to the culture. The growth is monitored by measuring the optical density at 610 nm every two hours for 26 h. The addition of KNO₃ by itself has no effect on the growth of this species of bacteria. The addition of L-arginine alone appears to actually slightly reduce growth, possibly due to pH effects. However, the addition of both ingredients together surprisingly promotes increased growth over the time of the experiment. The data is presented in Table 1 below:

TABLE 1

| Solution | Optical Density (610 nm) |
|---|---|
| Haemophilus | 1.02 |
| parainfluenzas only | |
| (Control) | |
| 0.75% L-arginine and | 0.65 |
| Haemophilus | |
| parainfluenzae | |
| 0.75% KNO₃ and | 0.99 |
| Haemophilus | |
| parainfluenzae | |
| *L-arginine, KNO₃, and. | 1.22 |
| Haemophilus | |
| parainfluenzas | |

*1.5% KNO₃ and 0.75% L-arginine, by wt. of the solution then added to the culture.

### Example 3—In Vitro Biofilm Model to Detect Changes in Nitrate Production

An in vitro model for oral biofilms is treated with either 3% KNO₃ or 1.5% L-arginine alone or in combination as simple solutions. Whole human saliva was used to inoculate sterile hydroxyapatite discs held in a vertical position using a specially designed steel lid as indicated in Kumar et al. (2019). Biofilms in Human Diseases: Treatment and Control Nature, the contents of which are incorporated herein by reference in their entirety.

Following 6 h of inoculum, biofilms are treated for 2 min with simple solutions, rinsed and returned to fresh, filter-sterilized saliva. Biofilms were treated 2 times per day, with approximately 6 h between treatments for the subsequent 3 days. On the fifth day, biofilms are treated one time in the morning and then harvested approximately 3 h after treatment by sonication. Bacterial pellets are frozen and later subjected to DNA extraction and sequencing of the V3-V4 region of the 16 s ribosomal RNA subunit in order to identify the relative abundance of bacteria present in each sample to the genus level. The final supernatants were also retained, filter-sterilized and tested for total nitrate/nitrite concentrations to determine if any changes in nitrate production could be detected from the biofilm communities.

First, salivary biofilms were treated with simple solutions of the active ingredients. For these studies, all solutions were made fresh in dH₂O and used within 1 h of being made. Solutions were prepared by weight to contain each of the following: 3% KNO₃, 1.5% L-arginine, both by weight of the solution, or no active. Additionally, all solutions contained 1.7% TSPP for additional stabilization. Solution pH values were not adjusted before treatment.

At the end of the experiment, filter sterilized samples were tested for total NO₃/NO₂ using a colorimetric nitrate/nitrite assay kit (Sigma). Due to low sample volumes, samples were only tested for total NO₃/NO₂.

TABLE 2

| | NO₃/NO₂ (nmole per |
|---|---|
| Sample | sample) |
| Untreated (control) | 5.93 |
| 1.5% L-arginine | 6.01 |
| 3% KNO₃ | 6.00 |
| 1.5% L-arginine + 3% | 7.06 |
| KNO₃ | |

While repeated treatment with L-arginine or KNO₃ alone leads to small increases in NO₃/NO₂ output, the combination of the two provides an even larger effect.

The composition of the microbiome of each sample is studied to assess the bacterial community shifts induced by treatment with: potassium nitrate alone, L-arginine alone, or potassium nitrate and L-arginine in combination. Focusing on the nitrate reducing species in each treatment sample, treatment with either ingredient (e.g., arginine or potassium nitrate by itself) alone shifts the proportions of these organisms in the final community. The greatest difference between untreated (PBS) and the KNO₃+L-arginine treated communities is increased relative to either active alone. In particular, Veillonella parvula, Haemophilus parainfluenzae and Rothia dentocariosa appear to be overrepresented in the dual active treated community. The results are demonstrated in Table 3 below:

TABLE 3

| | | 3% | 1.5% | 3% KNO₃ + |
|---|---|---|---|---|
| | Untreated | KNO₃ | L-arginine | 1.5% L-Arg |
| *Bacterial strain | (PBS) | Treated | Treated | Treated |
| Veillonella parvula | 4.59 | 0.78 | 3.28 | 10.24 |
| Haemophilus parainfluenzas | 0.14 | 0.04 | 0.83 | 0.98 |
| Neisseria flavescens subflava | 1.23 | 0.05 | 0.54 | 0.71 |
| Prevotella melaninogenica | 0.31 | 0.67 | 0.07 | 0.11 |
| Rothia dentocariosa | 0.02 | 0 | 0.02 | 0.12 |

*Results are given in relative units of abundance of bacteria present in each sample to the species level after bacterial pellets are frozen and subjected to DNA extraction and sequencing of the V3-V4 region of the 16s ribosomal RNA subunit.

To further explore the shift in the oral bacterial community, the in vitro biofilm assay is conducted using dentifrice slurries. The formulas of the dentifrices used in the slurries is detailed in Table 4. dentifrices containing 1.3% L-arginine and 3% KNO₃ alone or in combination and repeated the in vitro biofilm experiment described above, treating with a 1:1 slurry (remaining concentration of 1:1 slurry being e.g., about 1.5% KNO₃ and about 0.65% L-arginine) of toothpaste in water:

TABLE 4

| | Formula B | Control 3 | Control 4 |
|---|---|---|---|
| Ingredients | Paste | Paste | Paste |
| Water | Q.S. | Q.S. | Q.S. |
| | (e.g., ~6) | (e.g., ~6) | (e.g., ~6) |
| L-Arginine | 1.3 | — | 1.3 |
| Polyethylene glycol (e.g., | 2 | 2 | 2 |
| PEG 600) | | | |
| Xanthan Gum | 0.4 | 0.4 | 0.4 |
| Potassium Nitrate | 3 | 3 | — |
| Tetrasodium | 1.2 | 1.2 | 1.2 |
| Pyrophosphate | | | |
| Sorbitol | 58.1 | 61.1 | 59.4 |
| Silicas | 23 | 23 | 23 |
| Anionic surfactant | 1.5 | 1.5 | 1.5 |
| (e.g., sodium lauryl | | | |
| sulfate) | | | |
| Zwitterionic surfactant | 1.25 | 1.25 | 1.25 |
| (e.g., cocomidopropyl | | | |
| betaine) | | | |
| Flavors, Colors, and | 2 | 2 | 2 |
| other Minors | | | |
| Total | 100 | 100 | 100 |

The formulas described in Table 4 are used in an in vitro biofilm model that mimics 5 days' toothpaste usage twice daily. Saliva-derived biofilms were grown on hydroxyapatite discs held in a vertical position using a specially designed steel lid (ref). Sterilized discs are inoculated with 1.5 ml of 25% saliva in SHI medium and allowed to incubate for 4 h to allow for initial adhesion of bacteria. After 4 h, samples are treated for 2 min with 1:1 slurries of dentifrice:water and vigorously washed. Treated samples were transferred to fresh SHI medium and incubated overnight at 37° C., 5% CO₂. Samples are treated twice per day, with a minimum of four hours between treatments for the next 3 days. On the fifth day, samples are treated one time and then allowed to recover for at least 4 h in the incubator. Biofilms are harvested from discs by sonication and pellets were frozen and stored for further analysis via sequencing of the V3-V4 region of the 16 s ribosomal subunit.

The results of the assay with the dentifrice slurries are described in Table 5. The toothpaste treated samples appear to produce an expansion of the Veillonella parvula group in the presence of arginine alone or in conjunction with KNO₃. Similarly, the Neisseria flavescens and Haemophilus parainfluenzae groups are expanded. Interestingly, Rothia dentocariosa, a key nitrate reducing species, is only expanded in the presence of both arginine and KNO₃ but not either ingredient alone.

TABLE 5

| | | 1.3% | 3% | 3% KNO₃ + |
|---|---|---|---|---|
| | | L-arginine | KNO₃ | 1.3% L-Arg |
| | Untreated | Treated | Treated | Treated |
| *Bacterial strain | (PBS) | (Control 4) | (Control 3) | (Formula B) |
| Veillonella parvula_ | 6.13 | 18.12 | 5.41 | 20.98 |
| Haemophilus | 0.03 | 0.59 | 0.08 | 0.69 |
| parainfluenzas | | | | |
| Neisseria | 0.03 | 6.11 | 2.02 | 11.23 |
| flavescens subflava | | | | |
| Prevotella melaninogenica | 6.23 | 7.43 | 3.77 | 0.03 |
| Rothia dentocariosa | 0.01 | 0.03 | 0.03 | 0.11 |
| Rothia. mucilaginosa | 0.00 | 0.01 | 0.01 | 0.00 |

*Results are given in relative units of abundance of bacteria present in each sample to the species level after bacterial pellets are frozen and subjected to DNA extraction and sequencing of the V3-V4 region of the 16s ribosomal RNA subunit.

In the toothpaste treated samples, the Neisseria flavescens and Haemophilus parainfluenzae groups are also expanded. Interestingly, Rothia dentocariosa, a key nitrate reducing species, is only expanded in the presence of both arginine and KNO₃ but not either ingredient alone.

### Example 4—Measurement of Nitrite

Oral biofilms are grown on hydroxyapatite discs in saliva as discussed in the process detailed in Example 3. Over the course of five days, the biofilms receive treatment twice a day. Specifically, the biofilms receive treatment with samples containing simple solutions of water and 3% (by wt.) potassium nitrate and 5% (by wt.) arginine or a simple solution of water and 3% (by wt.) potassium nitrate alone. Following treatment, there is measurement of total nitrite (mg/L) in the biofilms following a five-minute challenge with 5% potassium nitrate.

Measurement of nitrite in biofilms indicates the following in Table 6:

TABLE 6

| | Biofilm Nitrite |
|---|---|
| Type of Treatment | Measurement (mg/L) |
| Untreated (control) | 0.11 |
| 3% (by wt.) potassium nitrate | 0.8 |
| 3% (by wt.) potassium nitrate | 4.7 |
| and 5% (by wt.) arginine | |

As demonstrated above in Table 6, there is an approximately 5.8-fold increase in the amount of nitrite following treatment with simple solutions containing 3% potassium nitrate and 5% arginine as compared to biofilm subject to treatment with simple solutions containing 3% potassium nitrate alone. This result indicates greater nitrate reduction following treatment with 3% potassium nitrate and 5% arginine as compared to biofilm subject to treatment with 3% potassium nitrate alone.

The present disclosure has been described with reference to exemplary embodiments. Although a limited number of embodiments have been shown and described, it will be appreciated by those skilled in the art that changes may be made in these embodiments without departing from the principles and spirit of the preceding detailed description. It is intended that the present disclosure be construed as including all such modifications and alterations insofar as they come within the scope of the appended claims or the equivalents thereof.

1. An oral care composition comprising:
(i) a water-soluble nitrate salt;
(ii) a basic amino acid in free or orally acceptable salt form;
(iii) water-soluble alkali metal polyphosphate alkali phosphate; and
(iii) more than 10% water, by weight of the composition.

2. The composition of claim 1 comprising a water-soluble nitrate salt is selected from an alkali or alkaline earth metal nitrate, or zinc nitrate, silver nitrate, or ammonium nitrate.

3. (canceled)

4. The composition of claim 1, wherein the nitrate salt is selected from lithium nitrate, sodium nitrate, potassium nitrate, magnesium nitrate, and calcium nitrate.

5. The composition of claim 4, wherein the nitrate salt is potassium nitrate.

6. The composition of claim 1, wherein the water-soluble alkali metal polyphosphate is selected from sodium pyrophosphate, potassium pyrophosphate, sodium tripolyphosphate and potassium tripolyphosphate.

7. The composition of claim 1, wherein the water-soluble nitrate salt is potassium nitrate and the water-soluble alkali metal polyphosphate salt is tetrasodium pyrophosphate.

8. (canceled)

9. The composition of claim 1, wherein the basic amino acid is arginine in free or orally acceptable salt form.

10. The composition of claim 1, wherein the composition comprises:
a. arginine;
b. potassium nitrate;
c. tetrasodium pyrophosphate;
d. more than 10% water, by weight of the composition.

11. The composition of claim 1, wherein the composition comprises:
a. 0.1-5% by wt. of potassium nitrate;
b. 1.65-2.25% by wt. of tetrasodium pyrophosphate;
c. 0.1%-5% by wt. of arginine; and
d. more than 10% water, by weight of the composition

12. The composition of claim 1, wherein the composition comprises a stannous ion source from 0.1 to 2% by wt. of the composition, wherein the stannous ion source is selected from stannous fluoride, stannous chloride or stannous pyrophosphate, or combinations thereof.

13. A method of treating or reducing blood pressure, wherein the method comprises administration of the composition according to claim 1 to the oral cavity of a subject in need thereof.

14. The method of claim 13, wherein the subject in need thereof has elevated blood pressure and/or is at risk for elevated blood pressure.

15. The method of claim 13, wherein the method is administered to a subject in need thereof in order to increase the presence of one or more oral bacteria in the oral cavity.

16. The method of claim 15, wherein the oral bacteria is selected from: Actinomyces naeslundii, Actinomyces odontolyticus, Actinomyces oris, Actinomyces viscosus, Bacillus brevis, Capnocytophaga sputigena, Corynebacterium durum, Corynebacterium matruchotii, Eikenella corrodens, Granulicatella adiacens, Haemophilus parainfluenzae, Haemophilus segnis, Microbacterium oxydans, Neisseria flavescens, Neisseria sicca, Neisseria subflava, Prevotella melaninogenica, Prevotella salivae, Priopionibacterium acnes, Rothia denticariosa, Rothia mucilaginosa, Staphylococcus epidermidis, Staphylococcus hemolyticus, Selenomonas noxia, Veillonella dispar, Veillonella parvula, Veillonella atypica, and combinations thereof.

17. (canceled)

18. (canceled)

19. (canceled)

20. (canceled)

21. (canceled)

22. The method of claim 13, wherein the subject in need thereof has low or reduced amounts of oral bacteria selected from: Actinomyces naeslundii, Actinomyces odontolyticus, Actinomyces oris, Actinomyces viscosus, Bacillus brevis, Capnocytophaga sputigena, Corynebacterium durum, Corynebacterium matruchotii, Eikenella corrodens, Granulicatella adiacens, Haemophilus parainfluenzae, Haemophilus segnis, Microbacterium oxydans, Neisseria flavescens, Neisseria sicca, Neisseria subflava, Prevotella melaninogenica, Prevotella salivae, Priopionibacterium acnes, Rothia denticariosa, Rothia mucilaginosa, Staphylococcus epidermidis, Staphylococcus hemolyticus, Selenomonas noxia, Veillonella dispar, Veillonella parvula, Veillonella atypica, and combinations thereof; and
wherein the amounts of oral bacteria are low or reduced relative to a reference standard.

23. (canceled)

24. (canceled)

25. The method of claim 13, wherein the subject in need thereof has low or reduced amounts of circulating nitric oxide, wherein the amount of circulating nitric oxide is low relative to a reference standard.

26. The method of claim 13, wherein the administration of the composition to the subject's oral cavity increases the amount of nitric oxide in the patient's blood plasma.

27. The method of claim 13 wherein the administration of the composition treats or reduces systemic blood pressure.

28. The oral care composition of claim 1, wherein the oral care composition can be ingested.

29. The method of claim 1, wherein the oral care composition can be ingested.
